(19) United States
(12) Patent Application Publication
(10) Pub. No.: US 2012/0288531 A1
(43) Pub. Date: Nov. 15, 2012

(54) PHARMACEUTICAL COMPOSITIONS FOR DELIVERY OF FERRIC IRON COMPOUNDS, AND METHODS OF USE THEREOF

(76) Inventors: Shmuel Tuvia, Netanya (IL); Dana Gelbaum, Tel Aviv (IL); Paul Salama, Ashdod (IL); Irina Karmeli, Maale Adummim (IL); Dori Pelled, Hod Hasharon (IL)

(21) Appl. No.: 13/349,320

(22) Filed: Jan. 12, 2012

Related U.S. Application Data
(60) Provisional application No. 61/432,790, filed on Jan. 14, 2011.
(60) Provisional application No. 61/537,364, filed on Sep. 21, 2011.

(51) Int. Cl. A61K 33/26; A61P 7/06; A61K 31/295; A61K 9/00; A61K 31/555

(52) U.S. Cl. 424/400; 424/647; 514/184; 514/502

(57) ABSTRACT

The pharmaceutical compositions described comprise a therapeutically effective amount of a ferric compound and at least one bioavailability enhancer for oral delivery. Some pharmaceutical compositions described herein include a suspension which comprises an admixture in solid form of a therapeutically effective amount of a ferric compound and at least one bioavailability enhancer (e.g. a salt of a medium chain fatty acid) and a lipophilic medium. The pharmaceutical compositions may be enteric-coated. Methods of treating or preventing diseases by administering such compositions to affected subjects are also disclosed. The methods of treatment described herein increase the level of iron in the bloodstream of a subject by administering to the subject an effective amount of an oral composition of a ferric iron compound.

### CLAIM OF PRIORITY

This application claims priority from U.S. Ser. No. 61/432,790, filed Jan. 14, 2011 and U.S. Ser. No. 61/537,364, filed Sep. 21, 2011, each of which is incorporated herein by reference in its entirety.

### FIELD OF THE TECHNOLOGY

The present invention relates to oral delivery of ferric iron compounds, formulations containing such compounds and methods of using such formulations.

### BACKGROUND

Iron is an essential component of every cell in the body. There are two forms of dietary iron: heme and non-heme. Heme iron is derived from hemoglobin and is found in animal foods that originally contained hemoglobin, such as red meats, fish, and poultry. Iron in plant foods such as lentils and beans is called non-heme iron, and this is the form of iron added to iron-enriched and iron-fortified foods. Heme iron is absorbed better than non-heme iron, but most dietary iron is non-heme iron. Without a sufficient supply of iron, hemoglobin cannot be synthesized and the number of erythrocytes in the blood cannot be maintained at an adequate level (reviewed in Geisser (2011) Pharmaceutics, 3:12-33.).

Anemia is the clinical manifestation of a decrease in circulating red blood cell mass, and usually is detected by low blood hemoglobin concentration. See National Kidney Foundation (2006) KDOQI Clinical Practice Guidelines and Clinical Practice Recommendations for Anemia in Chronic Kidney Disease. Am J Kidney Dis; 47:S1-S146 (suppl 3). The normal ranges for hemoglobin depend on the age and, beginning in adolescence, the gender of the person. Mild anemia may be defined as 9.5-13.0 g/dL for men (9.5-12 g/dL for women), moderate anemia as 8.0-9.5 g/dL and severe anemia as less than 8.0 g/dL.

Chronic kidney disease (CKD): Anemia is a common complication of declining renal function that contributes to the disease burden of chronic kidney disease (CKD). However, despite associations with adverse cardiovascular outcomes, end-stage renal disease, mortality and diminished quality of life, anemia remains poorly managed, with up to 70% of patients classed as anemic at the time of starting dialysis (reviewed in Macdougall (2010), Curr Med Res Opin., 26(2):473-482). Anemia develops early in the course of CKD and increases in frequency while the glomerular filtration rate (GFR) further declines (reviewed in Yilmaz et al (2011). Blood Purif., 32(3):220-225).

Iron deficiency is an important contributor to anemia in CKD, both in patients receiving chronic dialysis and in non-dialysis patients. The presence of either low iron stores (‘absolute’ iron deficiency), or inadequate iron available to meet the demand for erythropoiesis (‘functional’ iron deficiency), correlates significantly with reduced hemoglobin levels in non-dialysis-chronic kidney disease (ND-CKD) patients.

The goal of iron therapy in a patient with anemia and CKD is to achieve and maintain a target-range hemoglobin level. Iron agents may serve as primary therapy for selected patients (particularly those with ND-CKD) or as adjuvant therapy for those also undergoing treatment with an erythropoiesis-stimulating agent (ESA). Administered as adjuvants to ESAs, iron agents prevent iron deficiency and serve to minimize the dose of ESA needed to achieve target-range hemoglobin levels (see National Kidney Foundation, above).

In the US, the incidence and prevalence of kidney failure are increasing, outcomes are poor, and the cost is high. The prevalence of earlier stages of CKD is approximately 100 times greater than the prevalence of kidney failure, affecting almost 11% of adults in the US (National Kidney Foundation, above).
Iron supplementation treatment: The goal of iron supplementation treatment is not only to supply iron sufficient to correct the anemia but also to replenish iron body stores. Treatment options for iron supplementation include: (1) oral supplementation; and (2) intravenous supplementation:
(1) Oral supplementation: Although the oral route is the most convenient route of delivery for iron, in many cases it has serious limitations due to limited intestinal absorption of the iron and non-compliance, as follows. Almost all commercial oral preparations today use ferrous iron (Fe²⁺), although ferric iron (Fe³⁺) is the form of iron that binds to transferrin within the blood plasma, and this is the form of iron that the body can metabolize and use. However, ferric iron does not pass through the intestinal wall via the specific ferrous iron receptors. Ferrous iron passes through the intestinal wall via a receptor-mediated transcellular pathway and it is then converted to the ferric form whereby it is taken up by the protein transferrin in the bloodstream. Therefore, most oral iron supplements contain ferrous iron.

Physiological iron absorption in mammals (including humans, rats and dogs) is limited to the duodenum and proximal (upper) jejunum where the specific ferrous iron receptors are located; see for example Trinder et al. (2000) Gut 46: 270-276 and Christophersen et al (1976) Scand J. Gastroenterol. 11(4):397-402.

Ferrous iron when given orally has very low bioavailability. Because only small amounts are absorbed, large doses are necessary most of which is left non-absorbed in the intestine leading to side effects, which include digestive intolerance, causing nausea, heartburn, flatulence, abdominal pain, diarrhea or constipation, and black or tarry stools. Thus non-compliance of patients is very common because of this intolerance related to gastrointestinal adverse events.
(2) Intravenous supplementation: This is considered an important route for iron supplementation, and is indispensable in patients who are intolerant to oral iron or in whom current oral iron supplementation is not effective (e.g. CKD patients stage 3 and up), but it has many complications regarding administration and dosing. Today, patients in need of IV therapy typically receive several IV infusions of iron in hospital or out-patient clinics, to raise hemoglobin to normal levels (e.g. three infusions one week apart to upload about 1 gram of iron). The patients are then are left untreated, often for several months, until they become anemic again, and require another set of IV infusions to treat the anemia. Additionally, all intravenous iron products can lead to acute adverse reactions which can be minor to life-threatening, such as hypotension and anaphlactoid reactions (See Besarab and Coyne, above).

Enteric coating (also termed enteric film coating): In order to try to reduce the gastrointestinal side effects of oral iron delivery, some oral ferrous iron products have been enteric-coated. Enteric-coated products are designed to remain intact in the stomach and then to release the active substance in the intestine. It is commonly believed that enteric-coated dosage forms rapidly disintegrate on entry into the small intestine. However, this is not the case as there is a discrepancy between in vitro and in vivo performance of enteric coatings. For enteric-coated dosage forms in vitro, disintegration always occurs rapidly within few minutes in simulated intestinal pH. However, in vivo, it can take up to 2 h or more for the enteric-coated products to disintegrate after gastric emptying. As small intestinal transit time is of the order of 3-4 hours, disintegration and drug release from such enteric-coated dosage forms will occur in the distal small intestine. Thus the iron likely reaches a point in the intestine, past the duodenum and upper jejunum, where absorption is less efficient, leading to ineffective drug therapy; see Rudinskas et al (1989) CMAJ, 141, 565-566. It has been shown that enteric coated ferrous iron has on average only about 30% of the bioavailability of non-enteric coated products; see Walker et al (1989) CMAJ, 141, 543-547. Thus delayed drug release to the distal small intestine decreases the bioavailability of iron in conventional enteric-coated formulations, and such formulations may be ineffective. See Pharmacist's Letter/Prescriber's letter (2008) Detail-Document #20811, Therapeutic Research Center, Comparison of oral iron supplements; also Little (1999) Am Fam Physician 59:1598-604 and National Anemia Action Council (Nov. 6, 2008) A physician's guide to oral iron supplements.

Therefore, there is a need for an oral iron product to treat patients having mild, moderate and severe anemia who cannot be adequately treated with current products. For example, there is a need for an oral alternative suitable for advanced chronic kidney disease patients (stage 3 and up) or cancer patients or other individuals with serious illness who are recommended to switch to IV products. In particular, treatment of iron deficiency in non-dialysis-CKD patients can be challenging. There is a need for an oral iron product which can deliver, with minimal safety issues, amounts of iron to the blood which will be available to the body for use and creation of red blood cells and replenish of body iron stores. Such an oral iron could also reduce the amount of ESAs needed. There is a need for an oral iron preparation which does not have the GI side-effects of the current oral preparations. There is a need for an oral iron preparation which is a ferric iron preparation and which is formulated to pass through the intestinal wall and into the blood unaltered, for example via the paracellular route between the enterocytes and then can immediately be taken up by transferrin in the blood. There is a need for an oral iron preparation which may circumvent the problems of defective iron metabolism in certain illnesses; for example, there is a need for an oral iron preparation which is formulated to allow absorption of iron in a paracellular manner and not via the specific iron receptors.

### SUMMARY

The present invention relates to an oral formulation of ferric iron, which has novel and useful properties. This is achieved by incorporation of ferric iron in an oral delivery system which is preferably enteric-coated. This oral delivery is formulated to include a bioavailability enhancer to allow paracellular absorption of iron so that the iron does not need to be absorbed via specific iron receptors, which may have a defective mechanism in certain illnesses. Also disclosed herein are methods of oral delivery of ferric iron, for example, to treat a disorder described herein or to administer prophylactically to a subject, for example to delay or prevent the onset of a disorder described herein. In some embodiments, the method includes administration of a composition described herein in a treatment regime outside of a hospital setting (replacing an infusion protocol) and/or for chronic illness or prevention of illness.

In one aspect, the invention features an oral formulation of ferric iron compound and at least one bioavailability enhancer e.g. a medium chain fatty acid salt. As described above, also disclosed herein are methods of oral delivery of such formulations, for example, for the treatment or prophylactic treatment of a disorder described herein.

In one aspect, the present invention relates to a process for producing a pharmaceutical composition (oily suspension) which involves providing a solid powder of a therapeutically effective amount of a ferric compound and a solid powder comprising at least one bioavailability enhancer (e.g. a medium chain fatty acid salt) and optionally a solid powder comprising matrix forming polymer or matrix forming agent, and suspending the solid powders in a lipophilic medium, to produce an oily suspension containing in solid form the ferric compound and the medium chain fatty acid salt. The solid form may comprise a particle (e.g. consists essentially of particles, or consists of particles). The oily suspension may then be encapsulated in capsules which may be coated by an enteric coating and may be used for oral delivery. In another aspect of the invention the ferric compound and at least one bioavailability enhancer e.g. a medium chain fatty acid salt and optionally the matrix forming polymer or matrix forming agent, are solubilized in water, dried e.g. by lyophilization and the resulting solid is suspended in a lipophilic medium, to produce an oily suspension containing in solid form the ferric compound and the bioavailability enhancer e.g. medium chain fatty acid salt. This is shown in FIG. 1.

The present invention demonstrates delivery of the ferric iron compound to the intestine of rats, which is a model for oral delivery. Furthermore, the present invention demonstrates oral delivery to dogs, and the ferric iron compound is measured in the bloodstream with high bioavailability. The method of treatment described herein increases the level of iron in the bloodstream of a subject by administering to the subject an effective amount of an oral composition of a ferric iron compound.

The present invention may be used to treat or prevent anemia resulting from a disease or condition selected from anemia of chronic disease, e.g. chronic kidney disease (CKD) in particular stage 3 and up, and AIDS (caused by the HIV virus) and arthritis especially rheumatoid arthritis, inflammatory bowel disease such as Crohn's disease, cancer or where the subject is undergoing treatment with ESAs and/or with chemotherapy, celiac disease, autoimmune disease, hormone imbalances and endocrine deficiencies (such as hypothyroidism, male castration, Addison's disease, and herparathyroidism), surgery-related iron malabsorption e.g. post-gastrectomy or post-bariatric surgery or after removal of the duodenum and/or proximal jejunum (e.g. in Whipple procedure), not enough stomach acid, lack of intrinsic factor, hypoproliferative anemia including anemia of chronic disease, increasingly referred to as “anemia of inflammation” (which includes anemia of cardio-renal disease, the anemia of congestive heart failure, and anemia of Waldenstrom's macroglobulinemia), drug-induced anemia and hereditary anemia, menorrhagia and internal bleeding (e.g. from tumors, colon polyps, uterine fibroids, peptic ulcer or trauma), external bleeding (e.g. due to frequent blood donation, surgery, trauma or phlebotomy as treatment e.g. for plycythemia vera), various stomach and intestinal conditions (e.g. food sensitivity, parasitic infestation such as hookworms), cachexia (wasting syndrome), pregnancy and childhood anemia.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustrations and a further understanding of the various aspects and embodiments, and are incorporated in and constitute a part of this specification. The drawings, together with the remainder of the specification, serve to explain principles and operations of the described and claimed aspects and embodiments.

Throughout this application, various publications are referenced by author and year, and patents and applications including United States patents are referenced by number. The disclosures of these publications and patents and patent applications in their entireties are hereby incorporated by reference into this application in order to more fully describe the state of the art to which this invention pertains.

### BRIEF DESCRIPTION OF THE DRAWINGS

Various aspects of at least one embodiment are discussed below with reference to the accompanying Figures. For purposes of clarity, not every component may be labeled in every drawing. The Figures are provided for the purposes of illustration and explanation and are not intended as a definition of the limits of the invention. In the Figures:

FIG. 1 presents a process for production of a formulation of a ferric iron compound in accordance with one or more embodiments of the invention as referenced in the accompanying Examples.

FIG. 2 presents data showing administration of ferric iron compounds to Sprague-Dawley rats as described in Example 4.

### DETAILED DESCRIPTION

The present invention relates to an oral formulation of a ferric iron compound which has novel and useful properties. The current invention of an oral formulation of a ferric iron compound preferably protects ferric iron in the stomach because of an enteric-coated delivery system such as an enteric-coated capsule. The formulation includes a bioavailability enhancer which enables absorption along the length of the intestine, while dietary iron absorption is limited to the duodenum and proximal jejunum, because that is where the specific iron receptors are located. Without being bound by theory, the ferric iron in the formulations of the invention may pass through the intestinal wall via the paracellular route in between the enterocytes, and then may be immediately be taken up by transferrin in the blood.

To be more specific, non-heme dietary iron can be found in the intestines in two forms, ferrous ions and ferric ions. Most of the digested non-heme iron is in the ferric form (Fe3+), due to the low pH found in the stomach. Normally, luminal iron is enzymatically reduced to the ferrous iron form (Fe2+) in the duodenum and the proximal jejunum, prior to its uptake by specific iron receptors located in the enterocytes. The iron is then transferred through the intestinal wall to the blood, where the ferrous ion is re-oxidized to the ferric ion form. The formulations and methods described herein allow, for the first time, the enteral uptake of the ferric iron form to the blood without the use of the iron reduction-oxidation cycle. Without being bound by theory, this should produce less oxidative species which cause inter alia cardiovascular diseases.

Embodiments of the invention include a regimen of administration of capsules 1-4 times a day or even 1-3 times a week (if the iron loading in the formulation is sufficiently high) which can provide patients with favorable iron supplementation in small and frequent amounts. There are advantages (e.g. by providing a more stable blood level) in providing multiple small oral doses in contrast to the large amounts of infrequent intravenous dosage, normally every few months as described herein.
Pharmaceutical ferric compositions: The pharmaceutical compositions described herein include incorporation of a ferric iron compound as an active pharmaceutical ingredient (API), i.e. a therapeutic agent, within an oral dosage form which includes a bioavailability enhancer; the oral dosage is preferably enteric-coated. One embodiment of the invention is an oral dosage composition which comprises a therapeutically effective amount of a ferric iron compound and one or more bioavailability enhancers, which are enhancers of paracellular permeability in the small intestine; in other embodiments the oral dosage form is enteric coated; in other embodiments the oral dosage form is substantially free of a pyrophosphate compound (e.g. less than 1%); in other embodiments the oral dosage form is substantially free of vitamin C (ascorbate) (e.g. less than 0.1 mg, preferably less than 0.01 mg, vitamin C per 1.0 mg ferric iron compound); in other embodiments the oral dosage form is substantially free of a pyrophosphate compound (e.g. less than 10%, preferably less than 1% pyrophosphate compound); in other embodiments the oral dosage form is substantially free of talc (e.g. less than 10%, preferably less than 1%); in other embodiments of the oral dosage form the iron is not in a sustained release dosage form; in other embodiments the ferric ion is not chelated to a weakly basic anion exchange resin; in other embodiments the bioavailability enhancer is a medium chain fatty acid salt or derivative thereof.

One embodiment of the invention is an oral dosage composition which comprises a therapeutically effective amount of a ferric iron compound and one or more bioavailability enhancers wherein (A) the oral dosage form is enteric-coated; or (B) the ratio of the ferric iron compound to the total amount of bioavailability enhancer is in the range of 10:1 to 1:10 (or 4:1 to 1:4); or (C) the bioavailability enhancer is a medium chain fatty acid salt or derivative thereof.

One embodiment of the invention is an oral dosage composition, which comprises a therapeutically effective amount of a ferric iron compound and one or more surfactants. One embodiment of the invention is an oral dosage composition of ferric iron compound which comprises optionally a second and optionally a third (or more) therapeutic agent. One embodiment of the invention is an oral dosage composition which comprises only one therapeutically effective ferric compound. One embodiment of the invention is an oral dosage composition wherein the ferric compound is the sole therapeutically effective compound in the composition.

One embodiment of the invention is an oral dosage composition of the invention (comprising ferric iron compound) for the treatment of a subject who suffers from anemia and/or for increasing the level of iron in the bloodstream of a subject in need thereof. One embodiment of the invention is an oral dosage composition of the invention wherein the composition comprises a suspension which comprises an admixture of a lipophilic medium and a solid form wherein the solid form comprises a therapeutically effective amount of a ferric compound and one or more bioavailability enhancers; the bioavailability enhancer may be a medium chain fatty acid salt or derivative thereof. In another embodiment of the invention the oral dosage composition additionally comprises a matrix forming polymer or a matrix forming agent, which may be polyvinylpyrrolidone; preferably the polyvinylpyrrolidone is PVP-12 and/or has a molecular weight of about 3000.

The term “ferric iron compound” includes ferric iron in ferric salts and/or complexes including the following:
(a) ferric salts of carboxylic acids, e.g. ferric citrate, ferric tribasic citrate, ferric ammonium citrate, ferric tartrate, ferric acetylacetonate, ferric ammonium oxalate, ethylenediaminetetraacetate ferric sodium salt, ferric salts of mono-carboxylic acids (short, medium and long chains);
(b) ferric salts comprising an heterocyclic structure, e.g. ferric trimaltol and ferric hydroxy pyrones e.g. iron complexes of 3-hydroxy-4-pyrones; and
(c) other ferric derivatives, e.g. ferric inorganic salts such as ferric ammonium sulfate; ferric organic salts such as ferric dextrans, ferric trimaltose, ferric-hydroxide polymaltose, ferric acetyl-hydroxamate and ferric salts of amino acids.
   Particular ferric iron compounds are ferric ammonium citrate, ethylenediamine-tetraacetate ferric sodium salt (ferric sodium EDTA) and ferric acetyl hydroxamate.

In one aspect of the invention, a ferric iron compound and a medium chain fatty acid salt are in intimate contact or association with a substantially lipophilic (hydrophobic) medium. There may be an additional constituent for example a matrix forming polymer or a matrix forming agent, wherein the matrix forming polymer may be polyvinylpyrrolidone (PVP), cross-linked acrylic acid polymer (carbomer), polyvinyl alcohol polymer, hyaluronic acid and salts thereof, and cross-linked PVP (cross-povidones) inter alia. The ferric iron compound and the medium chain fatty acid salt or derivative thereof may be coated, suspended, sprayed by or immersed in a substantially lipophilic medium forming a suspension.

In one aspect of the invention the ferric compositions of the invention are oily suspensions and the amount of water in the compositions is very low. In another aspect of the invention the compositions incorporate a high amount (about 70-80% octanoic acid) and are also suspensions by visual analysis. A suspension of the invention may be a liquid suspension incorporating solid material, or a semi-solid suspension incorporating solid material (an ointment).

In some embodiments of the invention, the compositions comprise a suspension which comprises an admixture of a lipophilic medium and a solid form wherein the solid form comprises a therapeutically effective amount of a ferric compound and at least one salt of a medium chain fatty acid, and wherein the medium chain fatty acid salt is preferably present in the composition at an amount of 10% or more by weight, and optionally a matrix forming polymer and/or a matrix forming agent. The solid form may comprise a particle (e.g. consist essentially of particles, or consist of particles).

In some preferred embodiments, compositions of the invention include only excipients which are generally recognized as safe (e.g. GRAS), based on available data on human use, animal safety and regulatory guidelines. Some compositions of the invention may have other types of excipients (e.g. non-GRAS). In some embodiments, the compositions of the invention have amounts of excipients that are within the maximum daily doses as noted in such available data for each specific excipient.

The medium chain fatty acid salt may generally facilitate or enhance permeability and/or absorption of the ferric iron compound in the digestive system. The matrix forming polymer and/or matrix forming agent (see below) may serve to increase the effect of the bioavailability enhancer. In some embodiments the medium chain fatty acid salts include derivatives of medium chain fatty acid salts such as branched-chain fatty acid salts. The ferric iron compound, the medium chain fatty acid salt and the matrix forming polymer are in solid form, for example, a solid particle such as a lyophilized particle, granulated particle, pellet or micro-sphere. In some embodiments, the ferric iron compound, the medium chain fatty acid salt and the matrix forming polymer are all in the same solid form, e.g. all in the same particle. In other embodiments, the ferric iron compound and the medium chain fatty acid salt and optionally the matrix forming agent may be in a different solid form, e.g. each in a distinct particle.

In some preferred embodiments the compositions described herein provide a solid form comprising particles containing the ferric iron compound, which is then associated with the lipophilic medium. This is unlike emulsions, where water is an essential constituent of the formulation. The amount of water in these preferred embodiments is generally less than 6% by weight, usually less than about 3% or 2% or about 1% or less by weight and the water in the solid form is generally less than 4% by weight and usually less than 1% by weight.

The preferred embodiments described herein are suspensions, which comprise an admixture of a lipophilic medium and a solid form wherein the solid form comprises a therapeutically effective amount of a ferric iron compound, at least one salt of a medium chain fatty acid and preferably a matrix forming polymer. The solid form may be a particle (e.g. consist essentially of particles, or consist of particles). In some embodiments in the compositions described above, the solid form including the ferric compound also comprises one or more stabilizers of the ferric compound.

The amount of solid form (i.e. hydrophilic fraction) in the formulations of the invention is normally from about 10% to about 60%-70% or more of the formulation (w/w). In certain aspects of the invention, the amount of solid form is from about 20% to about 45%. In some embodiments, a bulking agent may be added.

The compositions of the invention can include a second therapeutic agent. Compositions of the invention which include a third (or more) therapeutic agent are also envisaged. The second and third (or more) therapeutic agent may be folate and/or magnesium and/or zinc and/or vitamin B12 and/or another active pharmaceutical ingredient.

In general, the composition may include from about 5% to about 50% or more by weight of the ferric iron compound e.g. about 5, 6, 7, 8, 9, 10, 15, 20, 25, 30, 35, 40, 45, or 50% or more by weight of the ferric iron compound. Also, in general, the composition may include from about 1% to about 10% or more by weight of elemental iron (the ferric ion) e.g. about 1, 2, 3, 4, 5, 6, 7, 8, 9, or 10% or more by weight of elemental iron.

In one aspect of the invention the pharmaceutical compositions described herein include incorporation of a ferric iron compound as an active pharmaceutical ingredient (API), i.e. a therapeutic agent, within an oral dosage form, which is preferably enteric coated and which includes a bioavailability enhancer.
Bioavailability enhancers: Preferred bioavailability enhancers are surfactants which act both as solubility promoters and transport promoters. In general, “solubility promoters” improve the ability of the ferric iron compounds to be solubilized in either the aqueous environment into which they are originally released or into the lipophilic environment of the mucous layer lining the intestinal walls, or both; “transport promoters” (which are frequently the same surfactants used as solubility promoters) are those which facilitate the ease by which the ferric iron compounds cross the intestinal wall (i.e. they facilitate uptake by transcellular or paracellular transport). Some particular bioavailability enhancers increase the bioavailability of the ferric iron compound in a formulation about 1.5, 2, 5, 10, 15, 20, 30 fold or more compared to the bioavailability of the ferric iron compound in a similar formulation without that bioavailability enhancer e.g. salts of medium chain fatty acids; some bioavailability enhancers (secondary bioavailability enhancers) are used in conjunction with a primary bioavailability enhancer and increase the bioavailability of the ferric iron by about 5, 10, 20, 30, 40, 50, 60, 70, 80, 90% or more compared to the bioavailability of the ferric iron compound in a similar formulation with the primary bioavailability enhancer but without the secondary bioavailability enhancer (e.g. lecithin and bile salts).

One or more bioavailability enhancers may perform one function only (e.g. solubility), or one or more bioavailability enhancers may perform the other function only (e.g. transport), within the scope of the invention. It is also possible to have a mixture of several bioavailability enhancers, some of which provide improved solubility, some of which provide improved transport (uptake) and/or some of which perform both.

Surfactants are believed to be useful both as solubility promoters and as transport promoters, for example, detergents. To reduce the likelihood of side effects, preferred detergents, when used as the bioavailability enhancers of the invention, are either biodegradable or reabsorbable, preferably biodegradable (e.g. biologically recyclable compounds such as bile acids, phospholipids, and/or acyl carnitines).

Preferred bioavailability enhancers include:
(i) medium chain fatty acid salts, in particular octanoate and decanoate salts such as sodium octanoate and sodium decanoate; derivatives of medium chain fatty acid salts such as branched chain fatty acid salts; medium chain fatty acids; derivatives of medium chain fatty acids such as mono-or di-glycerides and branched chain fatty acids; permeation enhancers described in U.S. Pat. No. 7,820,722, such as R₁—CH(R₂)-Q, where Q is a partially or completely neutralized —COOH functional group, R₁ is C₆₋₈ alkyl, R₂ is C₈₋₁₀ alkyl; acylcarnitines, acylcholines and acyl amino acids such as lauroylcarnitine, myristoylcarnitine, palmitoylcarnitine, lauroylcholine, myristoylcholine, palmitoylcholine, hexadecyl-lysine, N-acylphenylalanine, N-acylglycine;
(ii) bile salts in particular sodium salts of bile acids such as sodium taurocholate, sodium deoxycholate, sodium glycocholate, sodium chenodeoxycolate, sodium cholate, sodium lithocholate, sodium taurodeoxycholate, sodium ursodeoxycholate, sodium ursocholate, sodium dehydrocholate and sodium fusidate in particular sodium taurocholate;
(iii) non-ionic surfactants such as monoglycerides, a cremophore (polyethoxylated castor oil); polyethylene glycol fatty alcohol ethers, sorbitan fatty acid esters, polyoxyethylene sorbitan fatty acid esters, Solutol HS15, poloxamers, alkyl-saccharides (e.g. octyl glycoside, tetra decyl maltoside), polyoxyethylene ethers (e.g. Brij 36T, Brij 52, Brij 56, Brij 76, Brij 96, Texaphor A6, Texaphor A14, Texaphor A60), p-t-octyl phenol polyoxyethylenes (e.g. Triton X-45, Triton X-100, Triton X-114, Triton X-305) nonylphenoxypoloxyethylenes (e.g. Igepal CO series). Examples of monoglycerides are glyceryl monocaprylate (also termed glyceryl monooctanoate), glyceryl monodecanoate, glyceryl monolaurate, glyceryl monomyristate, glyceryl monostearate, glyceryl monopalmitate, and glyceryl monooleate; the commercial preparations of monoglycerides that are used also contain various amounts of diglycerides and triglycerides. Examples of sorbitan fatty acid esters include sorbitan monolaurate, sorbitan monooleate, and sorbitan monopalmitate (Span 40), or a combination thereof; particular examples of polyoxyethylene sorbitan fatty acid esters include Tween-20, polyoxyethylene sorbitan monooleate (Tween 80), polyoxyethylene sorbitan monostearate, and polyoxyethylene sorbitan monopalmitate;
(iv) ionic surfactants such as dioctyl sodium sulfosuccinate and lecithin phosphatidyl choline), cetylpyridinium chloride, and cholesterol derivatives;
(v) water soluble phospholipids e.g. lyso-phospholipids such as lysolecithin and lysophosphatidylethanolamine;
(vi) medium-chain glycerides which are mixtures of mono-, di- and triglycerides containing medium-chain-length fatty acids (caprylic, capric and lauric acids);
(vii) ethylene-diaminetetraacetic acid;
(iix) fatty acid derivatives of polyethylene glycol such as Labrasol® (caprylocaproyl macrogol-8 glycerides EP and caprylocaproyl polyoxyl-8 glycerides NF); Labrafil® (oleoyl macrogol-6 glycerides EP and oleoyl polyoxyl-6 glycerides NF); and Labrafac;
(iix) alkylsaccharides such as lauryl maltoside, lauroyl sucrose, myristoyl sucrose, palmitoyl sucrose and sucrose ester; and
(ix) salicylates such as sodium salicylate, 3-methoxysalicylate, 5-methoxysalicylate and homovanilate.

Compositions described herein can also include a combination of bioavailability enhancers and surfactants. Bioavailability enhancers are preferably present in a total quantity (including surfactants) that constitutes from about 5 to 50% by weight, preferably about 15 to 30% of the pharmaceutical composition.

In some embodiments of the invention, the compositions include a bioavailability enhancer e.g. the salt of a medium chain fatty acid or a derivative thereof in a solid form. For example, the salt of the medium chain fatty acid is in the form of a particle such as a solid particle. In some embodiments, the particle may be characterized as a granulated particle. In some embodiments, the solid form may generally result from a drying or evaporation process (e.g. spray-drying or lyophilization). For example, the therapeutic agent and the salt of the medium chain fatty acid can be prepared together by first preparing a solution such as an aqueous solution comprising both the ferric compound and the salt of the medium chain fatty acid and co-drying (e.g. co-lyophilizing) the solution to provide a solid form or particle that comprises both the ferric compound and the salt of the medium chain fatty acid (and other ingredients).

In one embodiment, the solid form of the hydrophilic fraction (solid particles) is formed by providing the powders of ferric compound, the bioavailability enhancer (e.g. sodium octanoate) and optionally a matrix-forming polymer (e.g. PVP-12), and not dissolving them in water but suspending them directly in the lipophilic medium. Thus there is no solubilization step and no drying step. This produces the WD (without drying) formulations described herein.

As described herein, in preferred embodiments the resulting solid particles (hydrophilic fraction) are associated with a lipophilic medium. For example, the solid particles may be suspended or immersed in a lipophilic medium.

In some embodiments the ratio of the weight of the ferric iron compound to the total weight of bioavailability enhancer is in the range of 10:1 to 1:10 or in the range of 8:1 to 1:8, or in the range of 5:1 to 1:5 or in the range of 4:1 to 1:4, or in the range of 3:1 to 1:3 or in the range of 2:1 to 1:2.

Medium chain fatty acid salts include those having a carbon chain length of from about 6 to about 14 carbon atoms. Examples of fatty acid salts are sodium hexanoate, sodium heptanoate, sodium octanoate (also termed sodium caprylate), sodium nonanoate, sodium decanoate (also termed sodium caprate), sodium undecanoate, sodium dodecanoate (also termed sodium laurate), sodium tridecanoate, and sodium tetradecanoate. In some embodiments, the medium chain fatty acid salt contains a cation selected from the group consisting of potassium, lithium, ammonium and other monovalent cations e.g. the medium chain fatty acid salt is selected from lithium octanoate or potassium octanoate or arginine octanoate or other monovalent salts of the medium chain fatty acids, or a combination thereof. In general, the amount of medium chain fatty acid salt in the compositions described herein may be from 2% up to about 50% by weight of the oily suspension. For example, in certain embodiments the medium chain fatty acid salt, preferably sodium octanoate, may be present at an amount of about 2%-50%, preferably about 11%-40%, preferably about 11%-28% and most preferably about 15% by weight of the oily suspension.

One embodiment of the invention comprises a composition comprising a suspension which consists essentially of an admixture of a lipophilic medium and a solid form wherein the solid form comprises a therapeutically effective amount of a ferric iron compound, at least one bioavailability enhancer, e.g. at least one salt of a medium chain fatty acid and a matrix forming polymer, and wherein the medium chain fatty acid salt is not a sodium salt. The salt may be the salt of another cation, e.g. lithium, potassium, ammonium or arginine.

Matrix forming polymer: In certain embodiments, the composition of the invention comprises a suspension which comprises an admixture of a lipophilic medium and a solid form wherein the solid form comprises a therapeutically effective amount of a ferric iron compound, at least one salt of a medium chain fatty acid and a matrix forming polymer. In certain embodiments, the composition comprises a suspension which consists essentially of an admixture of a lipophilic medium and a solid form wherein the solid form comprises a therapeutically effective amount of a ferric iron compound, at least one salt of a medium chain fatty acid and a matrix forming polymer. The matrix forming polymer is preferably present in the composition at an amount of about 0.5% to about 25% by weight, most preferably at an amount of about 1% to about 10% by weight.

Matrix forming polymers include polyvinylpyrrolidone (PVP) and cross-linked PVP (cross-povidones); ionic polysaccharides (for example, hyaluronic acid/hyaluronates and alginic acid/alginates); neutral polysaccharides (for example, dextran, methyl cellulose and hydroxypropyl methylcellulose (HPMC)); linear polyacrylic acid polymers including polymethacrylic acid polymers; cross-linked polyacrylic acid polymers (carbomers); amino-polysaccharides (e.g. chitosans); S-containing polymers (thiomers); and high molecular weight linear and bridged organic alcohols (for example, linear polyvinyl alcohol).

Carbomer is a generic name for cross-linked polymers of acrylic acid; carbomers may be homopolymers of acrylic acid, cross-linked with, for example, an allyl ether pentaerythritol, or allyl ether of sucrose or allyl ether of propylene or allyl sucrose or other sugars or allyl pentaerythritol or a polyalkenyl ether or divinyl glycol.

There are various forms of polyvinylpyrrolidone (PVP) polymers e.g. PVP-12, PVP-17 or PVP-25 (all may be obtained from BASF). PVP-12, PVP-17 and PVP-25 have average molecular weights of about 2500-3000, 10000 and 30000 respectively. In certain particular embodiments, the matrix forming polymer is PVP and the PVP is present in the composition at an amount of about 1% to about 20% by weight, or at an amount of about 3% to about 18% by weight, or at an amount of about 5% to about 10% by weight. In certain particular embodiments the PVP is PVP-12 and/or has a molecular weight of about 3000.

Trehalose and/or mannitol and/or other sugar derivatives may be substituted in certain embodiments instead of a matrix forming polymer and these are then termed “matrix forming agents”.

Hydrophilic fraction: In some embodiments of the invention, the above compounds, including the ferric compound, the bioavailability enhancer e.g. the medium chain fatty acid salt and optionally the matrix forming polymer (or substitute) are solubilized in an aqueous medium and then dried to produce a powder. The drying process may be achieved for example by lyophilization or by granulation or by spray-drying. The powder obtained is termed the “hydrophilic fraction”. In the hydrophilic fraction water is normally present at an amount of less than 6% by weight.

In a particular embodiment, the solid form of the hydrophilic fraction (solid particles) is formed by providing the powders of ferric iron compound, medium chain fatty acid salt and optionally matrix forming polymer (or substitute), and not dissolving them in water. The powders are then suspended in the lipophilic medium. Thus there is no solubilization step and no drying step. This produces the WD (“without drying”) formulations described herein.

Lyophilization may be carried out as shown in the Examples herein and by methods known in the art e.g. as described in Lyophilization: Introduction and Basic Principles, Thomas Jennings, published by Interpharm/CRC Press Ltd (1999, 2002) The lyophilizate (also termed lyophile) may optionally be milled (e.g. with a 150 micron mesh) or ground in a mortar. During industrial production the lyophilizate may be milled before mixing of the hydrophilic fraction and the lipophilic medium in order for example to control viscosity or for manufacturability or for other reasons.

Granulation may be carried out as shown in the Examples herein and by methods known in the art e.g. as described in Granulation, Salman et al., eds, Elsevier (2006) and in Handbook of Pharmaceutical Granulation Technology, 2^{nd} edition, Dilip M. Parikh, ed., (2005). Various binders may be used in the granulation process as described in the previous two references.

Spray-drying may be carried out by methods known in the art e.g. as described by Patel et al. (2009) Indian Journal of Science and Technology 2(10) 44-47 and by Shabde, Vikram (2006) Ph.D. thesis, Texas Tech University.

### Lipophilic (Hydrophobic) Medium:

Oil: As described above, in particular compositions of the invention described herein the ferric compound and the bioavailability enhancer (e.g. medium chain fatty acid salt) are in intimate contact or association with a lipophilic medium. For example, one or both may be coated, suspended, immersed or otherwise in association with a lipophilic medium. Suitable lipophilic mediums can contain, for example, aliphatic, olefinic, cyclic or aromatic molecules. Examples of a suitable aliphatic lipophilic medium include, but are not limited to, mineral oil, a paraffin, fatty acid monoglyceride, diglyceride, triglycerides, an ether, an ester, and combinations thereof. Examples of a suitable fatty acid are octanoic acid, decanoic acid and dodecanoic acid, also C7 and C9 fatty acids and di-acidic acids such as sebacic acid and suberic acid, and derivatives thereof. Examples of triglycerides include, but are not limited to, long chain triglycerides, medium chain triglycerides, and short chain triglycerides. For example, the long chain triglyceride can be castor oil or olive oil, and the short chain triglyceride can be glyceryl tributyrate and the medium chain triglyceride can be glyceryl tricaprylate or coconut oil. Monoglycerides are considered to be surfactants and are described below. Exemplary esters include ethyl isovalerate and butyl acetate. Examples of a suitable cyclic lipophilic medium include, but are not limited to, terpenoid-based compounds, cholesterol, cholesterol derivatives (e.g. cholesterol sulfate), and cholesterol esters of fatty acids. A non-limiting example of an aromatic lipophilic medium includes benzyl benzoate.

In some embodiments of the compositions described herein, it is desirable that the lipophilic medium includes a plurality of hydrophobic molecules. In some embodiments of the compositions described herein the lipophilic medium also includes one or more surfactants (see below). In some embodiments described herein the lipophilic medium comprises glyceryl tricaprylate or glyceryl tributyrate or castor oil or a mixture thereof.

Surfactants (Surface active agents): The oral dosage compositions of this invention can further include one or more surfactants. For example, as described above, in some embodiments a surfactant can be a component of the lipophilic medium and/or a surfactant can be a component of a solid form, or a surfactant can be in both. In some embodiments of the invention a bile salt may be incorporated in the solid form. Surfactants have been described above under bioavailability enhancers and include bile salts, lecithin, Tween 80 and monoglycerides such as glyceryl monocaprylate (GMC) or a combination thereof.

Compositions described herein including one or more surfactants (such as lecithin, bile salts, Tween 80) generally include less than about 12% by weight of total surface active agent (e.g. less than about 10%, less than about 8%, less than about 6%, less than about 4%, less than about 2%, or less than about 1%). In particular embodiments of the invention the total sum of all the surfactants is about 6-7% by weight in the composition. In certain embodiments the surfactants include Tween 80 at about 2% by weight and glyceryl monocaprylate at about 4-5% by weight in the lipophilic medium. In particular embodiments the surfactants include lecithin in the lipophilic medium; the lecithin can be at about 2-10%, preferably at about 6% by weight in the lipophilic medium.

The compositions of this invention may further contain a stabilizing agent, which may be located in the solid form and/or in the lipophilic medium; the stabilizer may stabilize the ferric iron, in particular it may maintain the ferric iron as ferric and prevent its reduction to the ferrous form. The compositions of this invention may further contain one or more viscosity adjusting substances.

### Methods of Making Pharmaceutical Compositions and the Compositions Described Herein.

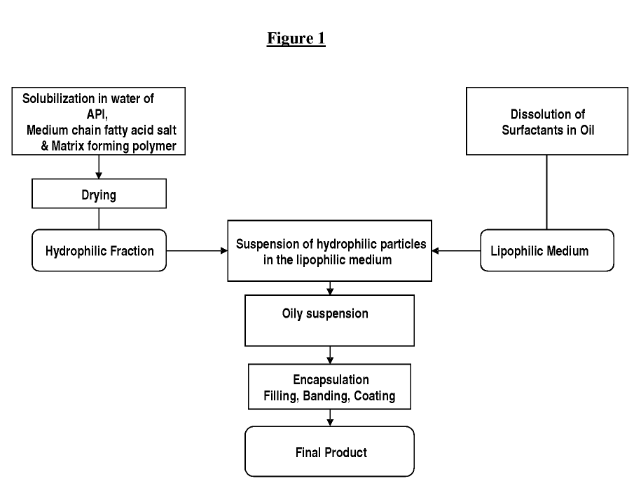

One embodiment of the invention is a process for producing a pharmaceutical composition which comprises preparing a water-soluble composition comprising a therapeutically effective amount of a ferric iron compound, a medium chain fatty acid salt and a matrix forming polymer or substitute such as matrix forming agent (as described herein) and preferably a surfactant, drying the water soluble composition to obtain a solid powder, and suspending the solid powder in a lipophilic medium, to produce a suspension containing in solid form the therapeutic agent, the medium chain fatty acid salt, surfactant and the matrix forming polymer (or matrix forming agent), thereby producing the pharmaceutical composition; in certain aspects of the invention the pharmaceutical composition contains about 10%-30% or more by weight of medium chain fatty acid salt; see FIG. 1.

One embodiment is a process for producing a pharmaceutical composition which comprises providing a solid powder of a therapeutically effective amount of a ferric iron compound and a solid powder comprising a medium chain fatty acid salt and optionally a solid powder comprising matrix forming polymer (or matrix forming agent), and suspending the solid powders in a lipophilic medium, to produce a suspension containing in solid form the therapeutic agent and the medium chain fatty acid salt, and optionally the matrix forming agent or matrix forming polymer, thereby producing the pharmaceutical composition. In certain aspects of the invention the composition contains 10-30% or more by weight of medium chain fatty acid salt. In certain aspects of the invention a surfactant as described herein is present, and it can be present in either the lipophilic medium or in the solid form or in both.

In an embodiment the formulation consists essentially of a suspension which comprises an admixture of a lipophilic medium and a solid form, wherein the solid form comprises a therapeutically effective amount of a ferric iron compound, preferably ferric ammonium citrate and about 10-40% preferably 15-30% of a medium chain fatty acid salt, preferably sodium octanoate, and about 0-30% or more, preferably 15-30% matrix forming polymer preferably PVP-12 and optionally 0.1-6% surfactant, preferably sodium taurocholate; and wherein the lipophilic medium comprises about 20-80%, preferably 30-70% triglyceride preferably glyceryl tricaprylate or glyceryl tributyrate or castor oil or a mixture thereof, and about 3-10% surfactants, preferably about 6%, preferably one or more of lecithin or glyceryl monocaprylate or Tween 80. In some embodiments, there may be less than 2%, or preferably less than 1% water in the formulation.

In another embodiment the formulation consists essentially of a suspension which comprises an admixture of a lipophilic medium and a solid form wherein the solid form comprises a therapeutically effective amount, preferably 20-40%, of a ferric iron compound, preferably ferric ammonium citrate and about 10-40% preferably 15-20% medium chain fatty acid salt preferably sodium octanoate, and about 0-30% matrix forming polymer preferably 5-15%, preferably PVP-12; and wherein the lipophilic medium comprises about 20-80%, preferably 30-60% triglyceride preferably glyceryl tricaprylate, about 3-10% surfactants, preferably about 6%, preferably lecithin. In some embodiments, there may be less than 2%, or less than 1% water in the formulation.

In particular embodiments the ferric iron compound is present at an amount of less than 40%, or less than 25%, or less than 10%, or less than 1% or less than 0.1%. In a particular embodiment the ferric iron compound is present at an amount of about 10% or 20% or 30% or 40% or 50% or more. In particular embodiments the ferric iron compound is selected from ferric ammonium citrate and ethylenediamine-tetraacetate ferric sodium salt (ferric sodium EDTA). In particular embodiments the ferric iron compound is ferric ammonium citrate and it is present at an amount of 10-40%, preferablyl5-30%.

In a particular embodiments of an oral dosage composition, the composition consists essentially of a therapeutically effective amount of a ferric iron compound (e.g. ferric ammonium citrate) and a medium chain fatty acid salt (e.g. sodium octanoate) suspended in a lipophilic medium (e.g. glyceryl tricaprylate), and this may additionally include a matrix forming polymer (e.g. PVP-12) or matrix forming agent and also one or more surfactants (e.g. lecithin and/or bile salts).

One embodiment of the invention relates to a process for producing a pharmaceutical composition (oily suspension) which involves providing a solid powder of a therapeutically effective amount of a ferric compound and a solid powder comprising at least one bioavailability enhancer (e.g. a medium chain fatty acid salt), and optionally a solid powder comprising matrix forming polymer or matrix forming agent, and suspending the solid powders in a lipophilic medium, to produce an oily suspension containing in solid form the ferric compound and the medium chain fatty acid salt. The solid form may comprise a particle (e.g. consists essentially of particles, or consists of particles). The oily suspension may then be encapsulated in capsules which may be coated by an enteric coating and may be used for oral delivery.

In the above formulations, the percentages recited are weight/weight and the solid form may be a particle (e.g. consist essentially of particles, or consists of particles).

Under normal storage conditions, the therapeutic agent (ferric iron) within the formulations of the invention is stable over an extended period of time, i.e. there is at least 90% preferably 95% ferric iron remaining after two years.

In certain embodiments the process produces a composition which consists essentially of a ferric compound, a medium chain fatty acid salt, surfactant and a lipophilic medium and optionally a matrix forming polymer. In embodiments of the invention the solid powder (solid form) consists essentially of a ferric iron compound, a medium chain fatty acid salt, surfactant and optionally a matrix forming polymer and/or a matrix forming agent. The compositions of this invention may further contain a stabilizing agent, which may be in the solid form and/or in the lipophilic medium.

Further embodiments of the invention are pharmaceutical compositions produced by the process described herein.

The ferric iron compound and/or medium chain fatty acid salt and/or matrix forming polymer, or any combination of therapeutic agent and other components, such as stabilizers or surfactants, can be prepared in a mixture which can be suspended in a lipophilic medium. Other components of the composition can also be added to the mixture. All components can also be added separately to be suspended in a lipophilic medium.

If desired, the pharmaceutical composition may also contain minor amounts of non-toxic auxiliary substances such pH buffering agents, and other substances such as for example, sodium acetate and triethanolamine oleate.

In some embodiments, the process for producing a pharmaceutical composition comprises providing a solid powder of a therapeutically effective amount of a ferric iron compound and a solid powder comprising a medium chain fatty acid salt and optionally a solid powder comprising matrix forming polymer or matrix forming agent, and suspending the solid powders in a lipophilic medium in a solution consisting essentially of octanoic acid, thereby producing the pharmaceutical composition.

Capsules and tablets: Preferred pharmaceutical compositions are oral dosage forms. Exemplary dosage forms containing the oily suspension include gelatin (hard gel or soft gel) or vegetarian capsules like starch or hydroxylpropyl-methylcellulose (“HPMC”) capsules; the capsules are enteric coated. Capsules which may be used to encapsulate the compositions of this invention are known in the art and are described, for example, in Pharmaceutical Capsules edited by Podczech and Jones, Pharmaceutical Press (2004) and in Hard gelatin capsules today—and tomorrow, 2nd edition, Steggeman ed., published by Capsugel Library (2002). An oral dosage form according to the invention comprises additives or excipients that are suitable for the preparation of the oral dosage form according to the present invention and may be prepared as described herein. Tablets comprising solid forms of the oily suspension, and tabletted with suitable excipients as known in the art, are also envisaged; the tablets are preferably enteric coated.

Enteric coated dosage forms are particular embodiments of the invention. Enteric coating can be applied to oral dosage forms, such as granules, pellets, capsules, or tablets. An enteric coating is resistant to stomach acid (thus protecting the ferric compound) and dissolves in the less acidic area of the intestines, thereby releasing the ferric compound. Thus an enteric coating can be termed a pH sensitive coating. Examples of enteric coatings are Acryl-EZE® (a methacrylic acid copolymer type C), Opadry™ Enteric series 91 (a polyvinyl acetate phthalate) Sureteric™ (also a polyvinyl acetate phthalate)—all from Colorcon; and Eudragit™ series (polymethylacrylates) from Evonik Rohm Gmbh. Capsules can be coated with the same enteric coating materials as tablets (sometimes a sub-coat or binder for better adhesion of enteric polymer is needed). A kit comprising instructions and the dosage form is also envisaged.

Sustained release dosage forms are designed to release an active pharmaceutical agent at a predetermined rate in order to maintain a constant drug concentration for a specific period of time with minimum side effects. This can be achieved through a variety of formulations, including liposomes and drug-polymer conjugates (e.g. hydrogels). Thus a sustained release formulation can also be termed “controlled release”. The oral dosage forms of ferric compounds exemplified herein are not sustained release formulations, but it is envisaged that the formulations of the invention could be modified to be sustained release formulations, if desired.

Additional formulations: The compositions of the invention may be formulated using additional oral dosage forms known in the art, for example as described in the following publications: Pharmaceutical Dosage Forms Vols 1-3 ed. Lieberman, Lachman and Schwartz, published by Marcel Dekker Inc, New York (1989); Water-insoluble Drug Formulation, 2^{nd} edition, Liu, editor, published by CRC Press, Taylor and Francis Group (2008); and Advanced Drug Formulation to Optimize Therapeutic Outcomes, Williams et al eds, published by Informa Healthcare USA (2008).

The compositions of the invention may be formulated using microparticulate technology for example as described in Microparticulate Oral Drug Delivery, Gerbre-Selassie ed., published by Marcel Dekker Inc (1994) and in Dey et al., Multiparticulate Drug Delivery Systems for Controlled Release, Tropical Journal of Pharmaceutical Research, September 2008; 7 (3): 1067-1075.

### Methods of Treatment:

One embodiment of the invention relates to a method for increasing the level of iron in the bloodstream of a patient in need thereof comprising administering to the patient an effective amount of an oral composition of a ferric iron compound; the composition is preferably enteric-coated. Another embodiment of the invention relates to a method of treating a subject suffering from a disease or disorder which comprises administering to the subject a composition of the invention in an amount sufficient to treat the condition i.e. a therapeutically active amount. Another embodiment of the invention relates to compositions of the invention for use in treating a disease or disorder or condition. Another embodiment of the invention relates to the use of an oral ferric iron compound in the manufacture of a medicament for the treatment of a disease or disorder or condition. Another embodiment of the invention relates to the use of an oral ferric iron compound in treatment of the following diseases or disorders or conditions, in particular treating or preventing the anemia resulting from these diseases or disorders or conditions: anemia of chronic disease e.g. CKD (in particular stage 3 and up) and AIDS (caused by the HIV virus) and arthritis especially rheumatoid arthritis, inflammatory bowel disease such as Crohn's disease, cancer or where the subject is undergoing treatment with ESAs and/or with chemotherapy, celiac disease, autoimmune disease, hormone imbalances and endocrine deficiencies (such as hypothyroidism, male castration, Addison's disease, and herparathyroidism), surgery-related iron malabsorption e.g. post-gastrectomy or post-bariatric surgery or after removal of the duodenum and/or proximal jejunum(e.g. in Whipple procedure), not enough stomach acid, lack of intrinsic factor, hypoproliferative anemia including anemia of chronic disease, increasingly referred to as “anemia of inflammation” (which includes anemia of cardio-renal disease, the anemia of congestive heart failure, and anemia of Waldenstrom's macroglobulinemia), drug-induced anemia and hereditary anemia, menorrhagia and internal bleeding (e.g. from tumors, colon polyps, uterine fibroids, peptic ulcer or trauma), external bleeding (e.g. due to frequent blood donation, surgery, trauma or phlebotomy as treatment e.g. for plycythemia vera), various stomach and intestinal conditions (e.g. food sensitivity, parasitic infestation such as hookworms), cachexia (wasting syndrome), pregnancy and childhood anemia. Cachexia is seen in patients with cancer, AIDS, COPD, MS, congestive heart failure, TB, familial amyloid polyneuropathy, mercury poisoning (acrodynia) and hormonal deficiency.

Some of these anemias, often termed refractory anemias, can be currently treated only by intravenous iron therapy. This invention provides advantages in providing adequate amounts of iron to replenish the iron deficiency by multiple small doses in contrast to the large non-frequent intravenous dosage, which has many disadvantages.

Without being bound by theory, in one aspect of the invention the compositions may be effective in treating or preventing anemia where the oral absorbance of iron is malfunctioning as a cause or as a result of the underlining illness, and/or where oral iron absorption is malfunctioning. This malfunction may be due to increased levels of hepcidin, which is a key regulator of iron metabolism. Such anemias are e.g. anemia of Waldenstrom's macroglobulinemia, of cardio-renal disease, of congestive heart failure, and anemias of chronic disease.

In another aspect of the invention the compositions may be effective in treating or preventing anemia in cases of increased demand for iron, as described above. Such cases include situations where the supply of iron must be increased due to certain physiological situations; e.g. infants and toddlers need more iron than older children and pregnant women also have higher iron needs, as do certain menstruating women (in cases of menorrhagia). In these cases levels of iron given by standards means is either not sufficient or causes side effects, in particular gastrointestinal distress.

The side effects of oral iron delivery by conventional methods make compliance a real issue in therapy, and these side effects are avoided due to the lower effective dosage needed by using formulations of the invention. One embodiment is a method of treatment of an anemic subject by a therapeutically effective amount of a ferric iron compound wherein the subject experiences less gastrointestinal side effects than when treated by a therapeutically effective amount of a commercial oral iron product; the subject might suffer 10-50% less gastrointestinal side effects. In some embodiments the method of treatment results in reduced gastrointestinal side-effects relative to commercial oral treatments. In another embodiment, a therapeutic dose of the invention results in 0-15%, preferably 0-10% gastrointestinal side-effects, opposed to about 20-70% gastrointestinal side effects when using a therapeutic dose of a commercial oral ferrous compound (see Macdougall, 2010; Rizvi et al (2011) Am J Gastroenterol 106: 1872-9).

This invention provides in one embodiment a method for increasing the level of iron in the bloodstream of a subject in need thereof comprising administering to the subject an effective amount of the oral dosage composition of the invention. This invention provides in one embodiment a method of oral treatment of mild, moderate and severe anemia by means of an oral ferric compound. The dosage regimen utilizing the ferric compounds is selected in accordance with a variety of factors including type, species, age, weight, sex and medical condition of the patient; the severity of the condition to be treated; and the particular compound or salt or complex thereof employed. An ordinarily skilled physician or veterinarian can readily determine and prescribe the effective amount of the drug required to prevent, counter or arrest the progress of the condition.

Oral dosages of the present invention, when used for the indicated effects, may be provided in the form of capsules each containing about 5, 10, 15, 25, 50,100, 200, mg or more of therapeutic agent or may be provided in the form of capsules each containing about 1, 2.5, 5, 10, 15, 25, or 50 mg or more of elemental iron (ferric ion).

Compounds of the present invention may be administered in a single daily dose, or the total daily dosage may be administered in divided doses of two, three, four, five or six times daily, or even 1-3 times a week (if the iron loading in the formulation is sufficiently high).

In some embodiments, the composition is administered at a daily dose of from about 10 mg/day to about 300 mg/day of therapeutic agent (ferric compound), administered once daily (e.g. in the morning or before bedtime) or twice or more daily (e.g. in the morning and before bedtime). In some embodiments one to four unit dosage forms (e.g. capsules) may be administered at one time. In some embodiments, the composition is administered at a daily dose of from about 10 to about 60 mg/day of elemental iron (ferric ion), e.g. about 10, 15, 20, 25, 30, 40, 50 or 60 mg/day of elemental iron. In the case of ferric ammonium citrate, the ferric ion is present at a level of about 14-40%, in particular 20%-23% of the weight of the ferric ammonium citrate.

The compositions described herein can be administered to a subject i.e. a human or an animal, in order to treat the subject with a pharmacologically or therapeutically effective amount of a therapeutic agent described herein. The animal may be a mammal e.g. a monkey, a mouse, rat, pig, dog, cat, horse, cow or sheep. As used herein the terms “pharmacologically effective amount” or “therapeutically effective amount” or “effective amount” means that amount of a drug or pharmaceutical agent (the therapeutic agent) that will elicit the biological or medical response of a tissue, system, animal or human that is being sought by a researcher or clinician and/or halts or reduces the progress of the condition being treated or which otherwise completely or partly cures or acts palliatively on the condition, or prevents development of the condition.

At least one embodiment of the invention is a method for increasing the level of iron in the bloodstream of a subject in need thereof, comprising administering to the subject an effective amount of an oral composition of a ferric iron compound. In at least one embodiment of the invention the bioavailability of the ferric iron compound is in the range 2%-70%, or 3%-9%, preferably 5% or 6% or 7% or 8% or 9% or 10% or 12% or 15% or more. The subject may be a human or animal.

One method of the invention may replenish depleted iron stores in a subject in need thereof, which in turn results in a rise in blood hemoglobin level in the amount of at least 1 g/dL within a fixed period, e.g.14-80 days; this may take place with or without concomitant therapy with ESA. In at least one aspect of the invention, in for example a maintenance regimen, additional treatments using the methods and oral dosage form of the invention may enable replenishing the increased (greater than 1 mg/day) daily loss of iron due to frequent blood sampling and/or occult gastrointestinal bleeding and/or other means of blood loss, and/or an increased rate of iron turnover to maintain the decreased red blood cell mass; normal daily loss of iron is about 1 mg/day. This increased rate of iron loss can occur for example in patients presenting with decreased duodenal iron absorption and/or decreased iron transport capacity because of a reduced transferrin concentration.

Sufficient iron should be administered to maintain transferrin saturation (TSAT) above 20% and preferably between 20% and 50%.

In some embodiments, a method can include treating a subject using an oral composition described herein who had previously been treated with an IV formulation of iron (e.g. ferric iron). For example, in some embodiments a method can include treating a subject with a maintenance dose of a ferric iron compound outside of a hospital setting. One embodiment is treatment of a subject who is first treated with a commercially available intravenous formulation of ferric iron, and who is subsequently switched to an oral regime comprising administering a composition described herein, which can be self-administered (e.g. as opposed to requiring administration by a health care professional). In some embodiments, a method can include treating a subject using an oral composition described herein who had previously been treated with an oral supplementation of iron (e.g. ferrous iron).

In at least one aspect, the compositions described herein improve bioavailability by enhancing the permeability of the intestinal epithelia to the ferric compound. For example, a composition described herein may facilitate absorption by permeating the intestinal epithelia primarily via unsealing of the tight junctions between intestinal epithelial cells (enterocytes), although it may also work by transcellular absorption.

All the percentage weights are relative to the overall weight of the oily suspension i.e. relative to the overall weight of the bulk drug composition, exclusive of the capsule and enteric coating.

The function and advantages of these and other embodiments will be more fully understood from the following examples. These examples are intended to be illustrative in nature and are not to be considered as limiting the scope of the systems and methods discussed herein.

### EXAMPLES

### Example 1

### Production Process of the Ferric Compound Formulations

The production process for the ferric iron compound formulations described in the following Examples and throughout the specification is essentially as described in above in the “Detailed Description” and in FIG. 1.

Below in Table 1 is one general example of a formulation of the invention:

TABLE 1

| | | Drug bulk |
|---|---|---|
| | | composition |
| | Ingredient | (% w/w) |
| Hydrophilic fraction | Ferric iron compound | 10.0 |
| (a solid form produced by | e.g. ferric citrate | |
| drying from the aqueous | PVP 12 | 10.0 |
| medium termed pre- | Sodium octanoate | 15.0 |
| hydrophilic fraction) | Water (from solid | 0.7 |
| | components) | |
| Lipophilic fraction | Surfactant e.g. GMC | 4.0 |
| (the hydrophilic fraction | Surfactant e.g. Tween | 2.0 |
| above is suspended in the | 80 | |
| lipophilic fraction) | Hydrophobic medium | 58.3 |
| | e.g. glyceryl | |
| | tricaprylate (GTC) | |

In some embodiments the solid form (described in Table 1) is produced by mixing the ferric iron compound, sodium octanoate and optionally PVP-12 as powders, and not dissolving them in water. Thus there is no solubilization step and no drying step. This produces the WD (“without drying”) formulations described in Example 5.

The complete formulation is an oily suspension (sometimes termed the bulk drug product). This oily suspension can then be encapsulated in an enteric-coated capsule for oral delivery.

### Example 2

### Selection of a Suitable Ferric Compound for Incorporation Into a Proprietary Oral Delivery Formulation

The following fifteen compounds were screened by various in vitro and in vitro tests, including stability data in intestinal fluids and in the formulation ingredients, to determine the most suitable ferric iron compound to incorporate into the oral formulation. These compounds include the following eight commercially available ferric compounds: ferric citrate (Sigma), ferric tribasic citrate (Fluka), ferric ammonium citrate (ammonium iron(III) citrate) (Fluka), ferric tartrate (Aldrich), ethylenediaminetetraacetic acid iron(III) sodium salt hydrate (Fluka), iron(III) acetylacetonate (Fluka), ammonium iron(III) oxalate (Sigma) and ferric dextran (ferric hydroxide dextran complex) (Sigma). Additionally, the following five ferric compounds were synthesized from the sodium salt of the corresponding counter-ion and ferric chloride: ferric gluconate, ferric glycinate, ferric lactate, ferric ascorbate and ferric aspartate; also ferric trimaltol and ferric octanoate were synthesized and partially purified.

After testing, the following compounds were not pursued: ferric tribasic citrate, ferric tartrate, ferric octanoate, ferric acetylacetonate and ferric citrate (because of solubility issues; also in the last two compounds there was presence of degradation product (Fe²⁺) at time zero which seems to increase with time); ferric ascorbate (because of fast reduction of Fe³⁺ to Fe²⁺⁾; ferric dextran complex (because of high molecular weight; also analytical method developed in-house cannot measure Fe³⁺ and Fe²⁺, probably due to strong complex formation between iron and dextran). Additionally, ferric ammonium oxalate (commercially available) and ferric gluconate (synthesized in-house) both had high ferrous iron (Fe2⁺⁾ contamination, in the preparations used, and so were not pursued (although this did not increase with time and this contamination may be less in a different preparation).

Based on the preliminary results it was decided to investigate further, using more stability tests, six compounds: ferric ammonium citrate, ferric sodium EDTA, ferric glycinate, ferric lactate, ferric aspartate and ferric trimaltol (although the last had solubility issues). The best results were obtained using ferric ammonium citrate and ferric sodium EDTA, which displayed good stability in the oily suspension for one month at 25° and 40° C.

### Example 3

### Preparation of Ferric Acetyl-Hydroxamate (Fe-AHA₃)

In addition to the screening of ferric iron compounds described in Example 2, it was decided to prepare and investigate ferric acetyl-hydroxamate. This complex was prepared by combination of FeCl₃ solution with acetohydroxamic acid (AHA) solution (1:3), followed by neutralization with NaOH and lyophilization, as shown in Table 2A. The final powder, in addition to Fe(III)-AHA₃ contains also NaCl.

TABLE 2A

| Ingredient | MW | Amount, g |
|---|---|---|
| FeCl₃*6H₂O | 270.3 | 2.70 |
| Water | — | 5.0 |
| Acetohydroxamic acid | 75.07 | 2.24 |
| (AHA) | | |
| Water | — | 5.1 |
| NaOH (6N) | 40 (240 mg/mL) | 4.9 mL (1.18 g) |

The lyophilizate produced (4.52 g) contained 11.83% Fe³⁺, Fe²⁺<0.1%. The complex was found to be stable for 4 hours in the pre-hydrophilic fraction of Table 1 (i.e. 10% PVP-12 and 15% NaC8). Neither precipitation nor Fe²⁺ formation was observed.

Two ferric acetyl-hydroxamate formulations were prepared, as shown in Table 2B.

TABLE 2B

Formulation

| | | B | C |
|---|---|---|---|
| | Ingredient | % w/w | % w/w |
| Hydrophilic | Ferric acetyl-hydroxamate | 10.0 | 10.0 |
| fraction | NaCl | 6.3 | 6.3 |
| (solid | PVP 12 | 10.0 | 10.0 |
| form) | Sodium octanoate | 15.0 | 24.0 |
| | Sodium taurocholate | 0.5 | 0 |
| | Residual water | 1.67 | 2.01 |
| Lipophilic | Tween 80 | 0 | 4.3 |
| medium | Glyceryl tricaprylate | 50.54 | 0 |
| | Lecithin | 6.0 | 0 |
| | Poloxamer F-68 | 0 | 1.7 |
| | Octanoic acid | 0 | 41.7 |

The above formulations were then tested in rats, and the bioavailability data are shown in Example 4.

### Example 4

### Ferric Iron Compound Formulations and Bioavailability Data

Several different formulations were prepared using separately both of the selected ferric iron compounds from Example 2, ferric ammonium citrate (FAC) and ferric sodium EDTA, and also ferric citrate and ferric acetyl-hydroxamate. The formulations are as shown below in Table 3. The method of production of the formulations was that described in Table 1 and FIG. 1, where the hydrophilic fraction was produced by drying an aqueous solution comprising FAC, sodium octanoate and PVP-12. In this case the drying was achieved by lyophilization.

TABLE 3

| | | Formulation | | |
|---|---|---|---|---|
| | | A | B | C |
| | Ingredient | % w/w | % w/w | % w/w |
| Hydrophilic | API | 10.00 | 10.00 | 10.00 |
| fraction | PVP 12 | 10.00 | 10.00 | 10.00 |
| (solid form) | Sodium octanoate | 15.0 | 15.0 | 24.0 |
| | Sodium taurocholate | 0 | 0.5 | 0 |
| | Residual water | 1.40 | 1.42 | 1.76 |
| Lipophilic | Tween 80 | 2.0 | 0 | 4.3 |
| medium | GMC | 4.00 | 0 | 0 |
| | Glyceryl tricaprylate | 57.60 | 57.08 | 0 |
| | Lecithin | 0 | 6.00 | 0 |
| | Poloxamer F-68 | 0 | 0 | 1.7 |
| | Octanoic acid | 0 | 0 | 48.24 |

The above formulations were tested in conscious rats as described in Example 6 below (animal models). The bioavailability (BA) results were calculated as absolute BA (compared to IV) per dose; see Table 4.

TABLE 4

| | | Mean | |
|---|---|---|---|
| | | AUC/dose | |
| | | (ng- | % BA |
| API | Formulation | h/mL/mg/kg) | (% CV) |
| Ferric ammonium | A | 8 | 15 (58) |
| citrate | B | 20 | 40 (33) |
| | C | 14 | 29 (54) |
| Ferric sodium | A | 10 | 8 (56) |
| EDTA | B | 22 | 18 (61) |
| | C | 4 | 3 (55) |
| Ferric citrate | A | 17 | 31 (90) |
| Ferric acetyl- | B | 9 | 5 (63) |
| hydroxamate | C | 11 | 6 (68) |

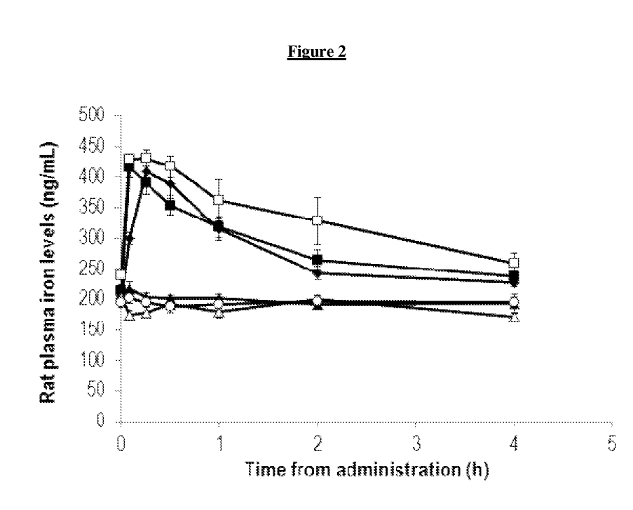

These results showed that all tested formulations showed bioavailability. A further series of experiments with several controls was then carried out; see FIG. 2. Six different groups of conscious Sprague-Dawley rats underwent administration of ferric compounds via three different routes as follows:

### Intravenously via Cannula at the Jugular Vein

(i) 30 mg/g ferric ammonium citrate in aqueous solution (2.1 mg elemental iron—closed squares);
(ii) 30 mg/g ferric dextran in aqueous solution (1.27 mg elemental iron—open squares);

### Per-Os (Gavage)

(iii) 26 mg/mL ferrous gluconate, an oral iron syrup (6.6 mg elemental iron—open triangles);

### Intra-Jejunal via Cannula at the Proximal Jejunum

(iv) 100 mg/g ferric ammonium citrate, formulated in formulation B of Table 3 (6.6 mg elemental iron—closed diamonds);
(v) 100 mg/g ferric ammonium citrate aqueous solution (6.6 mg elemental iron—closed triangles); and
(vi) 30 mg/g ferric dextran formulated in a formulation similar to that shown in Table 3, formulation B (12.7 mg elemental iron—open circles).

Blood samples were drawn via cannula in the jugular vein of each rat at baseline and at 5, 15 and 30 minutes and 1, 2, and 4 hours post-dosing. Pre- and post-dose rat plasma iron levels were next assayed using a colorimetric kit according to the manufacturer's instructions. The results, in FIG. 2, demonstrate that the rats absorbed from the jejunum only ferric ammonium citrate which was formulated. Intra jejunal delivery of both ferric ammonium citrate in solution and of formulated ferric dextran both gave baseline results, similar to those of ferrous gluconate (an oral iron syrup) given per os.

The ferric dextran used as control was produced from dextran of MW of about 5 kD, and once complexed with ferric hydroxide it forms particles with MW of about 100 kD; these are apparently too large to be absorbed from the jejunum.

### Example 5

### More Ferric Compound Formulations and Bioavailability Data

Further experiments were performed using formulations of ferric iron compounds:

(1) Several different formulations were prepared using ferric ammonium citrate (FAC). Two formulations are as shown below in Table 5, and were prepared as discussed in Example 4 where the drying step of the aqueous solution was achieved by lyophilization to produce the hydrophilic fraction (solid form).

Note that the 10% FAC formulation is formulation B of Table 3. The 20% FAC formulation differs from the 10% FAC formulation only in that there is twice the amount of FAC, and it was prepared using the same process. The formulations were tested in conscious rats as described in Example 6 below (animal models). The bioavailability (BA) results for the above two formulations were calculated as absolute BA (compared to i.v.) per dose. There was no significant difference between the two formulations regarding bioavailability; see Table 5.

TABLE 5

Formulation

| | 10% FAC | 20% FAC |
|---|---|---|
| Ingredient | % w/w | % w/w |
| FAC | 10.00 | 20.00 |
| PVP 12 | 10.00 | 10.00 |
| Sodium octanoate | 15.00 | 15.00 |
| Sodium taurocholate | 0.50 | 0.50 |
| Residual water (assumed | 1.42 | 1.82 |
| 4%) | | |
| Glyceryl tricaprylate | 57.08 | 46.68 |
| Lecithin | 6.00 | 6.00 |
| BA | 40 | 42 |
| % CV | 33 | 50 |

(2) Two 20% FAC formulations (D and E) were prepared as shown below in Table 6. The method of production of these formulations differed from that described in Table 1 and FIG. 1. Here, the hydrophilic fraction was produced by mixing powdered FAC, sodium octanoate and PVP (if used). Thus in this method there was no solubilization-in-water step and no drying step (no lyophilization). A control formulation (F) was also prepared (without sodium octanoate). Another formulation (G) was prepared by dissolving the powders in water. These four WD formulations (where WD=without drying) were tested in conscious rats as described in Example 6 below (animal models). The bioavailability (BA) results for the four formulations were calculated as absolute BA (compared to i.v.) per dose; see Table 6.

TABLE 6

WD Formulation

| | D | E | F | G |
|---|---|---|---|---|
| Ingredient | % w/w | % w/w | % w/w | % w/w |
| FAC | 20.0 | 20.0 | 20.0 | 20.00 |
| PVP 12 | 10.0 | 0 | 10.0 | 0 |
| Sodium octanoate | 15.0 | 15.0 | 0 | 15.0 |
| Glyceryl tricaprylate | 49.0 | 59.0 | 64.0 | 0 |
| Lecithin | 6.0 | 6.0 | 6.0 | 0 |
| % BA | 55 | 46 | 2.5 | 15 |
| % CV | 12 | 26 | 61 | 82 |

(3) The effect of repeated doses of formulated ferric ammonium citrate on iron pharmacodynamics was studied in normal SD rats. Ferric ammonium citrate was formulated as described in Formulation B in Table 3 above (6.3 mg iron per dose); the reference drug was ferrous sulphate (6.3 mg iron per dose). Conscious rats were used as described in Example 6 below (animal models). Rats were dosed daily for 21 days (18 doses) with the formulated ferric ammonium citrate (intra jejunum; n=15) or ferrous sulfate (gavage, per os; n=20). Blood samples were collected pre-dosing (Day 1) and post-dosing (Day 22). Blood hemoglobin was evaluated using colorimetric assay. The results are shown in Table 7.

TABLE 7

| | | Hb at | Hb at | |
|---|---|---|---|---|
| Formulation | | Day 1 g/dL | Day 22 g/dL | |
| tested | ROA | (Mean ± SE) | (Mean ± SE) | P |
| Formulated 10% | IJ | 13.4 ± 0.4 | 15.2 ± 0.2 | 0.0032 |
| FAC (6.3 mg iron) | | | | |
| Ferrous sulfate | PO | 14.2 ± 0.2 | 13.9 ± 0.2 | 0.1614 |
| (6.3 mg iron) | | | | |

The results in Table 7 were analyzed using non-paired Student t test in order to compare the treatment effect between groups. These calculations showed that there is a significant increase in the hemoglobin levels of the group treated with the formulated ferric ammonium citrate. No such increase was measured for the control group. These findings are in agreement with the single-dose observations in normal SD rats (Example 4, FIG. 2), in which the dosing of formulated ferric ammonium citrate rather than ferrous gluconate resulted in a marked effect on plasma iron levels.

### Example 6

### Animal Models for Testing the Activity of a Range of Different Ferric Compound Formulations

In order to test the capability of the formulation platform, the activity of formulations containing various different ferric iron compounds (APIs) is tested in one or more of the following animal models:
(i) jejunal administration to conscious (i.e. awake, non-anesthetized) rats; and
(ii) oral administration of capsules to large animals.

As controls for individual API metabolism, and for non-specific iron uptake, ferric and ferrous compounds were administered intra-jejunal, intra-venous and per os, to enable BA calculation.

### These Models are Described Below:

(i) Jejunal Administration to Conscious Rats

To test the activity of formulations in the jejunum of conscious rats, a specialized rat model was established in which two different cannulas are surgically implanted in male Sprague-Dawley or Lewis rats:
1—jejunal cannula to bypass the stomach and enable direct formulation administration to the jejunum; and
2—jugular vein cannula to determine the systematic levels of the administered dextran following jejunal administration.
   Rats were allowed to recover for 4 days before the study and were deprived of food for 18 hours before the start of the study.

Formulation containing ferric iron compound was administered to the jejunum of conscious rats, as described above, and separately saline solution containing the same ferric compound was administered intravenously as reference.

Blood samples were drawn from the jugular vein at an appropriate series of times post jejunal administration and post IV administration, plasma or serum was prepared and levels of iron were determined in each sample as described in Examples 4 and 6. The average absolute Bioavailability (aBA) achieved after jejunal administration of the formulation was then calculated.

The following pharmacokinetic parameters were calculated from the plasma iron concentrations after dosing: maximum plasma iron concentration (Cmax) and the time to Cmax (Tmax); area under the plasma iron concentration time curve from 0 to 240 or 1440 min (iron AUC₀₋₂₄₀ or AUC₀₋₁₄₄₀) using the linear trapezoidal method and the PK Solutions 2.0 computer program (SUMMIT Research Services, Ashland, Ohio, USA). Finally, the intra-jejunal API bioavailability was calculated per dose as proportion (% rBA) of the iron levels in blood after intravenous administration of the same API.

(ii) Oral Administration of Capsules to Large Animals

Oral experiments for pharmacokinetic (and toxicology) studies in large animals such as monkeys and dogs may be carried out as described in the following publications: Guidance for Industry, Investigators, and Reviewers: Exploratory IND Studies, (January 2006) U.S. F.D.A. Center for Drug Evaluation and Research (CDER); Pugsley et al (2010). J. Pharmacol Toxicol Methods. 62(1):1-3; and Mager et al (2009) Drug Metab Pharmacokinet 24(1): 16-24.

### Example 7

### Single-Dose Pharmacokinetic Study of Ferric Compounds Following Administration of Enteric-Coated Gelatin Capsule(s) to Male Beagle Dogs

The primary objective of this study was to determine iron bioavailability in beagle dogs following oral administration of ferric ammonium citrate (FAC) in the two formulations of Table 5, which were encapsulated in Acryl-EZE® coated gelatin size “0” gelatin capsules (i.e. enteric-coated capsules). Each capsule contained 60 mg or 120 mg ferric ammonium citrate, equivalent to 13.4 mg iron and 25.7 mg iron, respectively. (The slight difference in relative amounts of iron is because the amount of iron in the FAC varies from 20.5-22.5%, as per the manufacturer's specification).

Experiments were conducted in conscious male beagle dogs weighing between 9.4 to 12.6 kg. Food was withheld for 8-15 h prior to the study and returned after the completion of the intervention. Water was also withheld until 4 hr after drug dosing. The results are shown in Table 8.

Experiments were conducted in a crossover fashion whenever possible (N=3 for 12/16 of the tested dogs). Dogs were dosed with a single capsule or two capsules consisting of 60 mg or 120 mg ferric ammonium citrate respectively. In addition, 1 mL of 1.34 mg/mL FAC was dosed intravenously and an empty gelatin capsule was provided to four dogs as sham.

Relative bioavailabilities of FAC from experimental dosing regiments were compared to a reference 1.34 mg iron in water solution (pH 7.0) that was provided parentally through a peripheral vein. No side-effects were documented in any of the 16 tested dogs in this single-dose study.

Blood samples (approximately 1 mL/sample) were collected from the jugular vein at pre-dose and 0.5, 1, 1.5, 2, 2.5, 3, 3.5, 4, 4.5, 5, 6, and 8 hours post-dose in the oral groups and at the 0.083, 0.25, 0.5, 0.75, 1, 1.5, 2, 3, 4, 5, 6, and 8 hours post-dose in the injectable group. Serum iron from the samples was directly determined using a known method (acid reduction by hydrochloric acid and sodium ascorbate, using TPTZ [2,4,6-Tri-(2-pyridyl)-5-triazine] as the chromogen). To establish relative bioavailability, serum iron values determined on each baseline day were subtracted from those determined on the study day per dog. In addition, these iron levels were further adjusted to the mean baseline subtracted iron levels of the sham group. The following pharmacokinetic parameters were next calculated from these serum iron concentrations using standard computer programs: maximum serum iron concentration (Cmax) and the time to Cmax (Tmax); and the area under the plasma iron concentration time curve from 0 to 480 min (iron AUC,0-480). Finally, the reference FAC bioavailability was calculated per dose as proportion (% BA) of the iron levels in blood after intravenous administration.

TABLE 8

| | | AUC | | Calc. Iron |
|---|---|---|---|---|
| | Cmax | ng × h/mL | | Uptake |
| Test Article | ng/mL | (CV %) | % BA | (mg) |
| IV FAC | NA | 722 (26) | NA | NA |
| (1.34 mg iron in | | | | |
| water) | | | | |
| Oral formulated | 72 ± 15 | 336 (74) | 5.0 | 0.7 |
| FAC: 60 mg × 1 | | | | |
| (13.4 mg iron) | | | | |
| Oral formulated | 147 ± 21 | 714 (41) | 5.3 | 1.4 |
| FAC: 60 mg × 2 = | | | | |
| 120 | | | | |
| (26.8 mg iron) | | | | |
| Oral formulated | 124 ± 24 | 577 (68) | 4.4 | 1.1 |
| FAC: 120 mg × 1 | | | | |
| (25.7 mg iron) | | | | |
| Oral formulated | 230 ± 14 | 1359 (31) | 5.0 | 2.6 |
| FAC: 120 mg × | | | | |
| 2 = 240 | | | | |
| (51.4 mg iron) | | | | |

Mean serum iron concentration-time profile was determined for each iron treatment, as determined by subtracting pre-dose concentration from all other study day concentrations. The formulated FAC demonstrated approximately dose-linear pharmacokinetics, inasmuch as the AUC shown for the dosing of 120 mg FAC (one or two capsules) was approximately double the AUC shown for the dosing of the 60 mg FAC capsule. The iron uptake following FAC capsules resulted in iron uptake of 1.1-2.6 mg, depending on the number of capsules and iron loading. Such levels in human are considered therapeutically relevant since approximately 1 mg is the daily demand for iron in healthy humans, and up to approximately 5 mg of iron may be lost per day in patients with anemia of chronic disease such as chronic kidney disease (See Besarab and Coyne, above.)

Dog model bioavailability is considered predictive of human bioavailability for example see Khojasteh et al. (2011) Prediction of Human Pharmacokinetics, Chapter 7 in Drug Metabolism and Pharmacokinetics, Quick Guide, DOI 10.1007/978-1-4419-5629-3_{—}7,© Springer Science+Business Media, LLC.

### Example 8

### Measurement of Iron-Related Parameters

An iron panel usually consists of four tests. The first test is plasma iron measurement which measures the actual value of iron in the blood at the time of the test. The second test measures the hemoglobin level. The third test is for transferrin levels, or total iron-binding capacity; transferrin is a protein that carries iron in the blood, and in this test the iron saturation level of transferrin is calculated. The fourth test is serum ferritin measurement; ferritin is the primary protein used for iron storage in the body. Standard kits are used for these tests. The methods used are described inter alia in the following references: Punnonen et al (1997) Blood 89(3):1052-7; Koulaouzidis et al. (2009) Journal of Gastrointestinal and Liver Diseases 18:3: 345-352; Adler et al (1965) Analytical Biochemistry 11(2),159-163; Stookey (1970) Anal. Chem. 42:779-781; and Gibbs (1976) Anal. Chem. 48:1197-1200.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What we claim:

1. An oral dosage composition which comprises a therapeutically effective amount of a ferric iron compound and one or more bioavailability enhancers wherein the oral dosage form is enteric-coated; and wherein
a. the ratio of the ferric iron compound to the total amount of bioavailability enhancer is in the range of 10:1 to 1:10; or
b. the bioavailability enhancer is a medium chain fatty acid salt or derivative thereof.

2. The oral dosage composition of claim 1, wherein the ratio of the ferric iron compound to the total amount of bioavailability enhancer is in the range of 4:1 to 1:4.

3. The oral dosage composition of claim 1, wherein the bioavailability enhancers are enhancers of paracellular permeability in the small intestine.

4. The oral dosage composition of claim 1, which is substantially free of a pyrophosphate compound.

5. The oral dosage composition of claim 1, wherein the composition is substantially free of vitamin C (ascorbate).

6. The oral dosage composition of claim 1, wherein the iron is not in a sustained release dosage form.

7. The oral dosage composition of claim 1, wherein the composition is substantially free of talc.

8. The oral dosage composition of claim 1, wherein the ferric ion is not chelated to a weakly basic anion exchange resin.

9. The oral dosage composition of claim 1, wherein the ferric iron compound is selected from the group consisting of ferric salts of carboxylic acids, ferric salts comprising a heterocyclic structure; and other ferric derivatives.

10. The oral dosage composition of claim 9, wherein the ferric salt of carboxylic acid is selected from the group consisting of ferric citrate, ferric tribasic citrate, ferric ammonium citrate, ethylenediaminetetraacetate ferric sodium salt, ferric tartrate, ferric acetylacetonate, ferric ammonium oxalate and ferric salts of mono-carboxylic acids (short, medium and long chain).

11. The oral dosage composition of claim 9, wherein the ferric salt comprising an heterocyclic structure is selected from the group consisting of ferric trimaltol and ferric hydroxy pyrones e.g. iron complexes of 3-hydroxy-4-pyrones.

12. The oral dosage composition of claim 9, wherein the ferric salt comprising other ferric derivatives is selected from the group consisting of ferric inorganic salts such as ferric ammonium sulfate; and ferric organic salts such as ferric dextrans, ferric trimaltose, ferric-hydroxide polymaltose, ferric acetyl-hydroxamate and ferric salts of amino acids.

13. The oral dosage composition of claim 10, wherein the ferric salt is ferric ammonium citrate.

14. The oral dosage composition of claim 1, wherein the bioavailability enhancer is a medium chain fatty acid salt or derivative thereof.

15. The oral dosage composition of claim 14, wherein the medium chain fatty acid salt is sodium octanoate.

16. The oral dosage composition of claim 14, wherein the medium chain fatty acid salt is sodium decanoate.

17. The oral dosage composition of claim 1, which comprises optionally a second and optionally a third therapeutic agent.

18. The oral dosage composition of claim 1, which comprises only one therapeutically effective ferric compound.

19. The oral dosage composition of claim 1, wherein the ferric compound is the sole therapeutically effective compound in the composition.

20. The oral dosage composition of claim 1, for the treatment of a subject who suffers from anemia.

21. The oral dosage composition of claim 1, for increasing the level of iron in the bloodstream of a subject in need thereof.

22. The oral dosage composition of claim 20, wherein the anemia results from a disease or condition selected from: anemia of chronic disease e.g. chronic kidney disease (CKD), in particular stage 3 and up, and AIDS (caused by the HIV virus) and arthritis especially rheumatoid arthritis, inflammatory bowel disease such as Crohn's disease, cancer or where the subject is undergoing treatment with ESAs and/or with chemotherapy, celiac disease, autoimmune disease, hormone imbalances and endocrine deficiencies (such as hypothyroidism, male castration, Addison's disease, and herparathyroidism), surgery-related iron malabsorption e.g. post-gastrectomy or post-bariatric surgery or after removal of the duodenum and/or proximal jejunum, not enough stomach acid, lack of intrinsic factor, hypoproliferative anemia including anemia of chronic disease, referred to as “anemia of inflammation” (which includes anemia of cardio-renal disease, the anemia of congestive heart failure, and anemia of Waldenstrom's macroglobulinemia), drug-induced anemia and hereditary anemia, menorrhagia and internal bleeding, external bleeding, various stomach and intestinal conditions, cachexia (wasting syndrome) pregnancy and childhood anemia.

23-69. (canceled)
